(19) United States
(12) Patent Application Publication
(10) Pub. No.: US 2024/0352476 A1
(43) Pub. Date: Oct. 24, 2024

(54) SYNTHETIC PROMOTER FOR THE EXPRESSION OF HETEROLOGOUS PROTEINS IN PLANTS

(71) Applicant: TRANSACTIVA S.R.L., Udine (IT)

(72) Inventor: Piero Cristin, Gorizia (IT)

(73) Assignee: TRANSACTIVA S.R.L., Udine (IT)

(21) Appl. No.: 18/684,876

(22) PCT Filed: Aug. 12, 2022
(86) PCT No.: PCT/IT2022/050235

§ 371 Date: Feb. 20, 2024

(30) Foreign Application Priority Data
Aug. 20, 2021 (IT) 102021000022157

(51) Int. Cl. C12N 15/82; C07K 16/24

(52) U.S. Cl. CPC C12N 15/8258; C07K 16/241; C12N 15/8234; C07K 2317/13

(57) ABSTRACT

Synthetic promoter artificial DNA for the stable expression of heterologous proteins in plants, the sequence of which is optimized for the overexpression of the target protein in the internal endosperm.

### FIELD OF THE INVENTION

The present invention concerns a synthetic promoter for the stable expression of heterologous proteins in plants, which is applied in the field of plant molecular farming. In particular, the present invention is applied in the production of proteins of pharmaceutical interest in the endosperm of cereal and, specifically, of rice. The artificial sequence of the synthetic promoter described here has been optimized for the overexpression of the target protein in the internal endosperm.

More in particular, some embodiments described here concern a synthetic promoter artificial DNA for use in the stable expression of an antibody, in particular an anti-TNF alpha monoclonal antibody, even more in particular Infliximab, or derivatives thereof, in rice endosperm.

### BACKGROUND OF THE INVENTION

The seed-specific expression of pharmaceutical proteins in rice has numerous advantages: rice has a high yield of seed per unit of biomass; rice is an autogamous species and this drastically reduces the risk of contamination of food varieties; the cultivation, harvesting, processing and conservation processes make use of a centuries-old experience in the regions where this cereal has always played a primary role in the diet (Takaiwa et al., 2008); there are established protocols of stable transformation, also with a selectable positive marker or without a selectable marker, with which discrete levels of expression with marked tissue-specificity have been achieved; finally, the seed is a natural accumulation and reserve organ of the plant. In particular, the possibility of inducing the expression in a specific manner in the internal endosperm makes rice an exploitable bioreactor from an industrial point of view. In fact, this part of the seed is not particularly rich in other proteins, lipids and other contaminants, but mainly consists of water-insoluble starch. After the seed has been harvested, it is possible to separate the endosperm from the rest of the aleuronic layer using techniques that are already usually used for refining food rice.

Many efforts have been made to investigate the mechanisms that influence the expression level of a recombinant protein (Qu and Takaiwa, 2004; Qu et al., 2008). Whatever the plant species or tissue considered, the two main factors that influence the accumulation of a protein are the synthesis speed and the degradation speed. The synthesis speed is mainly influenced by the transcription speed, the stability of the mRNA and the translation speed. The transcription speed is greatly determined by the efficiency of the promoter, but the number of copies of T-DNA and the genomic position in which the individual T-DNAs integrate are equally important (Takaiwa et al., 2008). The degradation speed depends on the intrinsic stability of the protein and the localization of the accumulation (Egelkrout et al., 2012). In this sense, the seed-specific expression is a factor that drastically reduces the degradation speed. In addition to tissue-specificity, an important role for the accumulation and above all for the stability of the protein must be attributed to subcellular localization; in fact, the compartment to which the nascent protein will be sent will influence its folding, the possible assembly of various subunits, the level and type of glycosylation (Twyman et al., 2003).

The complex system formed by the endoplasmic reticulum in the maturing seed has great efficiency in processing nascent proteins thanks to the abundance of molecular chaperones. Various signal peptides at the N-terminal with lengths ranging from 18 to 30 amino acids are able to send the protein to which they are linked to the lumen of the reticulum. The addition of a signal peptide is therefore necessary for the accumulation in seed. The absence or removal of the signal peptide leads to an undetectable expression even when the gene is transcribed at high levels (Sharma et al., 2000; Yang et al., 2002; Takagi et al., 2005b).

Another important factor for the level of expression is the stability of the mRNA. The half-life of this family of molecules ranges from a few minutes to several days depending on the presence of specific motifs present in the sequence. The sequence close to the m7Gppp (5′-cap) structure and the poly-A tail can occur in a variety of forms that stabilize the transcript in a different way. It is also known that the entire sequence not translated in 5′ is fundamental for the recruitment of translation factors (Liu et al., 2010), as well as in some cases of transcription (De Amicis et al., 2007). Another factor that could significantly increase the translation speed of a transcript is the optimization of the codon dialect based on the host organism. In fact, the frequency with which the various codons for each amino acid appear varies from organism to organism and may be related to the relative abundance of tRNAs for the amino acid in question (Streatfield et al., 2001).

Currently, in order to obtain the endosperm-specific expression of a transgene, it is put under the control of reserve protein promoters of the rice seed. Plants store a considerable quantity of their reserves of nitrogen, sulfur and carbon in the form of seed reserve proteins, which are subsequently used in the post-germination phase of development. Based on their solubility, these reserve proteins are divided into three classes: globulins, prolamins and glutelins. The globulins, characterized by their solubility in saline solutions, serve as the main reserve of nutrients in the embryo of the seeds of mono-and dicotyledonous plants, while the prolamins, soluble in alcohol, and the relatively insoluble glutelins, perform this function only in the endosperm of monocotyledons. Two of the most widespread classes of globulins, designated 7S and 11S on the basis of their sedimentation properties, are present in different proportions among dicotyledons (Okita et al., 1989). Unlike most cereals that use prolamins as a nitrogen reserve, the most abundant proteins in rice seed are glutelins. These proteins, which can make up 80% of endosperm proteins, are synthesized and accumulated in the intermediate phase of tissue development (Yamagata et al., 1982). Prolamins are also present in rice seed, but this fraction amounts to only 5-10% of the total proteins. The synthesis of glutelins and prolamins is not coordinated. Glutelins begin to be synthesized 4-6 days after flowering, while prolamins begin to be detectable only several days later. The localization of these two families of proteins occurs in morphologically distinct protein bodies which are the result of two different cellular processes (Tanaka et al., 1980; Krishnan et al., 1986). Glutelins are encoded by a small multigenic family of about 11 genes per haploid set (Okita et al., 1989; Takaiwa et al., 1987;

Takaiwa et al., 1990; Takaiwa et al., 1991). These genes are divided into three subfamilies designated GluA, GluB and GluC, consisting respectively of 5, 5 and 1 gene per haploid genome respectively (Qu et al., 2008). Already at the end of the 1980s, promoters of two rice glutelin subfamilies had been sequenced and tested in vitro (Okita et al., 1989). In the early 2000s, research on the promoters that induce the expression of these proteins intensified, with a view to their possible exploitation in the field of biotechnology. By comparing promoters of various rice reserve proteins based on the level of expression of the uidA reporter gene, encoding the enzyme beta-glucuronidase of E. coli (GUS), it was deduced that the most potent promoter is that of 26 kDa globulin (Wu et al., 1998), an unexpected result given that this reserve protein represents only 2-8% of the seed protein. This was confirmed by a transient expression experiment in the endosperm of cultured rice; the 26 kDa globulin promoter proved to be more active than the promoters of three different glutelins and one prolamin (Hwang et al., 2001a). Furthermore, in a stable transformation experiment of rice plants, the gene that encodes lysozyme was introduced under the control of a series of glutelin promoters and of the globulin promoter. In this case too, the globulin promoter proved to be more effective (Hwang et al., 2002). It should be noted that two-dimensional electrophoresis experiments confirmed the existence of a single form of globulin (Pan and Reeck, 1988), while Southern Blotting analyses carried out on the rice genome indicated the presence of only one copy of the gene encoding this protein (Shorrosh et al., 1992); on the contrary, there are about ten genes encoding for as many forms of glutelin. From the whole of the tests carried out, it can be concluded that there is no significant correlation between the abundance of a class of reserve proteins and the strength of the corresponding promoters.

The seed-specific promoters can be divided into groups according to various criteria. Not only does the level of expression vary drastically from one promoter to another, but also the temporal and spatial profile of expression differ from case to case (Qu and Takaiwa, 2004; Qu et al., 2008).

Using the localization of the expressed protein as the association criterion, it is possible to distinguish promoters:
specific for the internal endosperm (26 kDa globulin, 14 kDa and 16 kDa allergens);
specific for the subaleuronic layer (mainly glutelins);
specific for the peripheral aleuronic layer (mainly prolamins);
active both in the aleuronic and in the embryo (oleosins, embryo globulin);
active both in the scutellum and in the endosperm (PPDK, AlaAT) (Qu and Takaiwa, 2004).

Taking into consideration the relative transcriptional activity, the promoters classified as strong are:
the glutelin B4 promoter (Qu and Takaiwa, 2004);
the 26 kDa globulin promoter (Qu and Takaiwa 2004);
the 10 kDa and 16 kDa prolamins promoters (Qu and Takaiwa, 2004);
the glutelin C promoter (Qu et al., 2008).

Understanding of the mechanisms that regulate specific expression in seed has grown very much in recent years. Despite the amount of information available, there is still no qualitative leap in the production of recombinant proteins in seed. Therefore, the technological platform for production based on plant organisms still suffers from limitations due to insufficient biomass yield. What the pharmaceutical industry requires in order to abandon traditional production systems, such as cultured cells, in favor of plant production, is an increase in the yield of recombinant protein per unit of biomass to be processed. Only in this way can the extraction and purification costs be reduced, which, as is known, make up the majority of the economic burden in the case of green bioreactors: indeed, the production of biomass has a very limited cost. Many proteins of industrial and pharmaceutical interest have been produced in rice seed (Evangelista et al., 1998) and these potentials for synthesis further increase the need to address and resolve the problem of specific yield.

Many natural promoters have been characterized and are currently available to direct the expression of transgenes in rice seed. Most of them direct expression in the aleuronic or sub-aleuronic layer. From a careful study of the literature, only the glutelin B4 promoter, the glutelin B5 promoter, the glutelin C promoter, and the globulin promoter allow the accumulation of recombinant protein in the internal endosperm. Of these, the glutelin B4 promoter allowed to obtain the best results (Qu and Takaiwa 2004). Its use has been described in the international application WO-A-2009/112508 for the production of a lysosomal enzyme and in the international application WO-A-2018/189764 for the production of an antibody, both in the name of the present Applicant. However, the range of promoters of seed reserve proteins has now been extensively investigated and no alternatives have emerged that can guarantee a higher level of expression.

The document Piero Cristin, 2013, PHD thesis, pp. 1-219 describes the expression of an enzyme in rice endosperm, in particular glucocerebrosidase (GCase).

There is therefore a need to perfect a synthetic promoter artificial DNA for the stable expression of heterologous proteins in plants, in particular in rice endosperm, which can overcome at least one of the disadvantages of the state of the art.

In particular, one purpose of the present invention is to provide a synthetic promoter artificial DNA for use in the stable expression of an antibody, in particular an anti-TNF alpha monoclonal antibody, more particularly Infliximab, or its derivatives, in rice endosperm.

The Applicant has devised, tested and embodied the present invention to overcome the shortcomings of the state of the art and to obtain these and other purposes and advantages.

### SUMMARY OF THE INVENTION

The present invention is set forth and characterized in the independent claims, while the dependent claims describe other characteristics of the invention or variants to the main inventive idea.

The embodiments concern synthetic promoter artificial DNA for the stable expression of heterologous proteins in plants, in particular in rice endosperm.

In particular, the embodiments concern a synthetic promoter artificial DNA for use in the stable expression of an antibody, in particular an anti-TNF alpha monoclonal antibody, more in particular Infliximab, or its derivatives, in rice endosperm.

The artificial DNA comprises, in the 5′→3′ direction, a plurality of DNA fragments coming from three specific natural endosperm promoters, respectively the gutelin B4 promoter, hereafter GluB4, the prolamine 16 kDa promoter, hereafter Prol16, and the globulin-1 (26 kDa) promoter, hereafter Glb. These fragments are operatively connected to each other by means of two synthetic linking and spacing portions interposed between the fragments. A first linking portion is an artificial sequence with a length comprised between 75 and 100 nucleotides and a second linking portion is an artificial sequence with a length of at least 100 nucleotides, in particular comprised between 100 and 125 nucleotides.

In accordance with some embodiments, the first linking and spacing portion is defined by SEQ ID N°: 3.

In accordance with some embodiments, the second linking and spacing portion is defined by SEQ ID N°: 5.

In accordance with some embodiments, a first of the fragments as above, in the 5′→3′ direction, is a sequence of 82 nucleotides, present in the initial part of Glb and containing the REB binding sites for the transcription factor RISBZ2, defined by SEQ ID N°: 1.

In accordance with some embodiments, a second of the fragments as above, operatively connected directly downstream of the first fragment, in the 5′→3′ direction, is a sequence of 208 nucleotides present in the initial part of GluB4 and defined by SEQ ID N°: 2. The first linking and spacing portion is operatively connected directly downstream of the second fragment, in the direction 5′→3′.

In accordance with some embodiments, a third of the fragments as above, operatively connected directly downstream of the first linking portion, in the 5′→3′ direction, is a sequence coming from Prol16, in particular containing the two cis-regulatory motifs GCN4 and prolamin-box. The second linking and spacing portion is operatively connected directly downstream of the third fragment, in the 5′→3′ direction.

In accordance with some embodiments, the third fragment is a sequence of 118 nucleotides present in the intermediate part of Prol16 and defined by SEQ ID N°: 4.

In accordance with some embodiments, a fourth of the fragments as above, operatively connected directly downstream of the second linking portion, in the 5′→3′ direction, is a sequence of 484 nucleotides which constitutes the second half, or final part, of Glb and contains Glb cis-regulatory motifs, defined by SEQ ID N°: 6.

In accordance with some embodiments, a fifth of the fragments as above, operatively connected directly downstream of the fourth fragment, in the 5′→3′ direction, is a sequence of 425 nucleotides which constitutes the final part of GluB4, including the TATA-box motif and the transcription start site, which together induce the start of mRNA synthesis on the fused DNA template downstream of the promoter, defined by SEQ ID N°: 7.

In accordance with some embodiments, the synthetic promoter artificial DNA described here is a sequence defined by SEQ ID N°: 10.

Another aspect of the present invention is artificial DNA defined by SEQ ID N°: 3 or by SEQ ID N°: 5 with linking and spacing function in a synthetic promoter.

Yet another aspect of the present invention is artificial DNA defined by SEQ ID N°: 9, with the function of enhancer for a synthetic promoter.

Other embodiments concern an expression vector comprising synthetic promoter artificial DNA in accordance with the present description.

Other further embodiments concern a method for the stable production of recombinant proteins in plants, in particular for the stable production of an antibody, more in particular an anti-TNF alpha monoclonal antibody, even more in particular Infliximab, or its derivatives, in rice endosperm. In some embodiments, the method comprises:
transformation of plants using an expression vector as described here,
processing of the transformed seed with industrially scalable methods,
extraction and purification of the protein of interest.

In some embodiments, the stable production of recombinant proteins in plants described here is in rice endosperm.

The present invention, as expressed by the embodiments described above, allows to implement the performances of the rice seed-specific expression system through genetic engineering interventions on the promoter sequence. The engineering of natural sequences to obtain synthetic seed-specific promoters according to the present description requires combining levels of transcriptional activity greater than those found in natural promoters with the maintenance, at the same time, of a high degree of tissue-specificity.

Consequently, the present invention achieves a synergistic effect, thanks to the spatial association in a single sequence of portions of artificial DNA deriving from promoters of different seed reserve proteins. The Applicant has experimentally selected portions of natural promoters containing real or presumed cis-regulatory motifs responsible for the specificity of expression. Particular attention was paid to the context in which these motifs were combined. The outcome of the analysis and experimentation conducted by the Applicant is a synthetic sequence with the function of seed-specific promoter that exceeds the best natural promoters in level of expression. This result was obtained without losing the seed-specificity.

As regards the applicative outcomes of the synthetic promoter described here, it is advantageously inserted in a technological platform able to exploit plant organisms as bioreactors for the production of recombinant biopharmaceuticals. This platform has been optimized by the Applicant by intervening on all the mechanisms that regulate the expression of a transgene in a complex plant organism. The ultimate goal is to integrate the progress made in relation to every single element that affects performance into the expression system, keeping the technological platform at a cutting-edge level.

Practical applications of the promoter according to the present invention, which are described below, have led to a doubling of the expression level compared to what was possible to achieve with the reference promoter, that is, the glutelin B4 natural promoter. Despite the increase in level, the expression of recombinant protein remained relegated to the internal endosperm. The doubling of the desired concentration of protein in the biomass translates into halving the size of the plant intended for the purification of the active ingredient, which reduces by up to 50% the costs related to the phase of the production process that is by far the most onerous, that is, the so-called downstream phase.

The combination of fragments derived from different natural promoters of seed reserve proteins, linked together by synthesis sequences, has the purpose of simultaneously recruiting seed-specific transcription factors which, as a rule, separately activate the expression of different seed reserve proteins. The result obtained confirmed significantly increasing the expression level. The experiments conducted by the Applicant, and described in detail below, have highlighted a doubling of the expression level compared to the more powerful specific endosperm promoter of natural origin that has been studied so far. The synthetic promoter of the present invention has, however, maintained the specificity characteristics of its natural analogues. Advantageously, no expression of the transgene was found outside the internal endosperm.

### DESCRIPTION OF THE DRAWINGS

These and other aspects, characteristics and advantages of the present invention will become apparent from the following description of some embodiments, given as a non-restrictive example with reference to the attached drawings wherein:

FIG. 1 is a graph that shows the histograms relating to the expression level of the single primary transforms with pTRS/SGlbB4-STE::GLA::NOSter, in black, and with pTRS/GluB4-STE::GLA::NOSter, in gray;

FIG. 2 is a graph that shows the overlap of the histograms relating to the expression level of the single primary transforms with pTRS/SGlbB4-BRU::LC-IFX::NOSter/SGlbB4-BRU::HC-IFX::NOSter, in black, and with pTRS/GluB4-BRU::LC-IFX::NOSter/GluB4-BRU::HC-IFX::NOSter, in gray;

FIG. 3 is a chromatogram of an affinity chromatography of a batch of Infliximab from transgenic rice transformed with the vector pTRS/SGlbB4-BRU::LC-IFX::NOSter/SGlbB4-BRU::HC-IFX::NOSter. On the right, the UV absorbance scale at 280 nm, corresponding to the curve described by a solid line in the graph. On the left, the pH scale, corresponding to the curve described by the dotted line in the graph. The curve described by the dashed line corresponds to the conductance trend;

FIG. 4 is a graph that shows the light chain sequence coverage from the combination of the data of all the proteolytic digests (residues 1-214);

FIG. 5 is a graph that shows the heavy chain sequence coverage from the combination of the data of all the proteolytic digests (residues 1-160);

FIG. 6 is a graph that shows the heavy chain sequence coverage from the combination of the data of all the proteolytic digests (residues 161-240);

FIG. 7 is a graph that shows the heavy chain sequence coverage from the combination of the data of all the proteolytic digests (residues 241-320);

FIG. 8 is a graph that shows the heavy chain sequence coverage from the combination of the data of all the proteolytic digests (residues 321-450);

FIG. 9 is a histogram that summarizes the results of the analysis of the glycan species, with Rice-IFX in black and control (Rice-RTX) in gray.

We must clarify that in the present description the phraseology and terminology used, as well as the figures in the attached drawings also as described, have the sole function of better illustrating and explaining the present invention, their function being to provide a non-limiting example of the invention itself, since the scope of protection is defined by the claims.

It is understood that elements and characteristics of one embodiment can be conveniently combined or incorporated into other embodiments without further clarifications.

### DESCRIPTION OF SOME EMBODIMENTS

Some embodiments described here concern a synthetic promoter for the expression of heterologous proteins in plants, in particular in cereal endosperm and, in a specific example, rice endosperm.

In particular, some embodiments concern a synthetic promoter artificial DNA for use in the stable expression of an antibody, in particular an anti-TNF alpha monoclonal antibody, more in particular Infliximab, or its derivatives, in rice endosperm.

This synthetic promoter has a specific artificial DNA sequence, advantageously optimized for the overexpression of recombinant proteins, in particular monoclonal antibodies as indicated above, in cereal endosperm, in particular rice endosperm.

Another aspect of the present invention is a method that provides to use such sequence for the production of recombinant proteins, in particular monoclonal antibodies as indicated above, in cereal endosperm, and for example rice endosperm.

In one specific implementation, the synthetic promoter artificial DNA described here is defined by a sequence of 1519 nucleotides, which activates the massive transcription of the downstream DNA segment. Activation is restricted in space and time, in a manner that can completely overlap with what happens with a specific endosperm promoter of natural origin. The promoter sequence described here provides a unique association of fragments coming from three specific natural endosperm promoters and from two completely synthetic portions with linking and suitable spacing purposes. The fragments of natural origin present in the synthetic promoter described here derive from three specific endosperm promoters, assigned to control as many seed reserve proteins. The three promoters are listed in table 1 below.

TABLE 1

| | Promoter sequence | |
|---|---|---|
| Reserve protein | accession | Abbreviation |
| glutelin B4 | AY427571 | GluB4 |
| Prolamin 16 kDa | AY427574 | Prol16 |
| Globulin-1 (26 kDa) | AY427575 | Glb |

Here and in the present description, the promoters will be identified with the abbreviations shown in the third column of table 1 above.

The embodiments described here therefore concern a synthetic promoter artificial DNA for the stable expression of heterologous proteins in plants, in particular in cereal endosperm. This artificial DNA comprises, in the 5′→3′ direction, a plurality of artificial DNA fragments, in particular five, for example, coming from three specific natural endosperm promoters, respectively GluB4, Prol16 and Glb, operatively connected to each other by means of two synthetic linking and spacing portions interposed between the fragments.

In some embodiments, the first linking and spacing portion is an artificial sequence with a length comprised between 75 and 100 nucleotides and the second linking and spacing portion is an artificial sequence with a length of at least 100 nucleotides, in particular comprised between 100 and 125 nucleotides.

According to one embodiment, in the artificial DNA the first and the penultimate fragment, in the 5′→3′ direction, come from Glb and the second and last fragment, in the 5′→3′ direction, come from GluB4. The third fragment coming from Prol16 is disposed intermediate, in the 5′→3′ direction, between the first and the second fragment on one side, and the fourth (penultimate) and fifth (last) fragment on the other side, with the first linking and spacing portion disposed between the second fragment and the third fragment and the second linking and spacing portion disposed between the third (intermediate) fragment and the fourth (penultimate) fragment.

Therefore, according to one embodiment, the artificial DNA comprises, in the 5′→3′ direction, a first fragment coming from Glb directly connected downstream to a second fragment coming from GluB4. The latter is directly connected downstream to the first linking and spacing portion which, in turn, is connected downstream with a third fragment coming from Prol16. The second linking and spacing portion is directly connected downstream of the third fragment. A fourth fragment, coming from Glb, is connected directly downstream of the second linking and spacing portion and a fifth fragment coming from GluB4 is connected directly downstream of the fourth fragment.

According to some embodiments, the artificial DNA consists of the first fragment, the second fragment, the first linking and spacing portion, the third fragment, the second linking and spacing portion, the fourth fragment and the fifth fragment, thus disposed in the 5′→3′ direction.

In one possible implementation, the first and fourth fragments coming from Glb are different from each other. The first fragment comes from the initial part of Glb, while the fourth fragment comes from the final part of Glb.

In one possible implementation, the second and fourth fragments coming from GluB4 are different from each other. The second fragment comes from the initial part of GluB4, while the fifth fragment comes from the final part of GluB4.

In one possible implementation, the artificial DNA consists exclusively of said plurality of fragments and of said two synthetic linking and spacing portions interposed between said fragments.

The GluB4 promoter is the starting point for constructing the synthetic promoter. This was modified by replacing and inserting DNA segments coming from the Prol16 and Glb promoters interspersed with the two artificial portions. The result is a synthetic sequence that combines in the appropriate context, salient regulatory elements for high expression in the endosperm.

The choice of the natural promoters from which to extract the fragments, which fragments of each promoter to recombine and the way in which these are combined are an integral part of the present invention. Briefly, the rationale was to:
start from promoters with complementary expression profiles from the spacetime point of view (Qu et Takaiwa, 2004; Qu et al., 2008);
choose the fragments of each promoter on the basis of the presence, proven or supposed, of cis-regulatory elements that are determinant for the expression in endosperm (internal experimental data);
combine the fragments along the sequence, each at the optimal distance from the transcription start site and in order to obtain a synergistic effect.

In order to obtain the result referred to in the last point, the two completely synthetic portions with linking and appropriate spacing purposes were developed. Precisely for this reason, not only the choice, but above all the disposition and the distance between the elements are part of the present invention.

Starting from the 5′ end, in some embodiments the elements of the synthetic promoter described here are:
1. a first fragment defined by a sequence of 82 nucleotides present in the initial part of Glb and containing the REB binding sites for the transcription factor RISBZ2 (SEQ ID N°: 1);
2. a second fragment defined by a sequence of 208 nucleotides present in the initial part of GluB4 (SEQ ID N°: 2);
3. a first linking and spacing portion defined by an artificial sequence, linker1, with a length comprised between 75 and 100 nucleotides having a synthetic linker function (SEQ ID N°: 3);
4. a third fragment defined by a sequence of 118 nucleotides present in the intermediate part of Prol16 (SEQ ID N°: 4);
5. a second linking and spacing portion defined by an artificial sequence, linker2, with a length comprised between 100 and 125 nucleotides, having a synthetic linker function (SEQ ID N°: 5);
6. a fourth fragment defined by a sequence of 484 nucleotides which constitutes the second half, or final part, of Glb and containing various cis-regulatory motifs typical of this promoter (SEQ ID N°: 6);
7. a fifth fragment defined by a sequence of 425 nucleotides which constitutes the final part of GluB4, including the TATA-box motif and the transcription start site, which together induce the start of the messenger (mRNA) synthesis on the fused DNA template downstream of the promoter (SEQ ID N°: 7).

The association between the sequences SEQ ID N°: 6 and SEQ ID N°: 7, already gives rise on its own to a very strong and at the same time compact synthetic promoter (just 900 bp), identified as SEQ ID N°: 8. This association had already been experimentally identified by the Applicant as a basis for constructing a synthetic promoter. The key to obtaining a further increase in the expression level was the fusion in 5′ of a synthetic enhancer element specially designed for this purpose. This is the complex defined by SEQ ID N°: 9 and identified here as a whole as fragment S. This fragment S consists of the sequences SEQ ID N°: 1, SEQ ID N°: 2, SEQ ID N°: 3, SEQ ID N°: 4 and SEQ ID N°: 5. Of particular importance is the length and linker2composition (SEQ ID N°: 5). This is preferably at least 100 bp long.

The sequences SEQ ID N°: 1, SEQ ID N°: 2 and SEQ ID N°: 4, within the respective natural promoters of origin, are located at a distance of several hundred nucleotides from the transcription start site. For this reason they have been recombined to form the end 5′ of the synthetic promoter, with the specific intent of achieving an enhancing effect on the part located downstream and closest to the transcription start site. Within these sequences there are cis-regulatory elements that have been chosen and disposed with the aim of achieving a synergistic effect on the expression level.

For example, in SEQ ID N°: 4 there are two cis-regulatory motifs: GCN4 and prolamin-box. These two cis-regulatory motifs form the typical endosperm-box: an association with a synergistic effect widespread in many rice-specific endosperm promoters (Yamamoto et al., 2006). It is typically found between 150 and 300 nucleotides upstream of the transcription start site. In Prol16, on the other hand, it is found particularly distant from the transcription start site, about 400 nucleotides upstream. Despite this, Prol16 is a strong specific endosperm promoter. Furthermore, the fusion of the endosperm-box of Prol16 upstream of the fragment coming from the Glb promoter has shown, in experiments conducted by the Applicant, to increase the expression level thereof. For this reason, in the synthetic promoter described here, the fragment containing the endosperm-box was fused in a position that is distant from the transcription start site. At the same time, it is spaced from both the elements upstream as well as from the elements downstream, by means of the two synthetic linking and spacing portions. The length of the two synthetic linkers (SEQ ID N°: 3 and SEQ ID N°: 5) was designed to prevent interference between the various cis-regulatory motifs, while maintaining the synergistic effect.

The sequence indicated in SEQ ID N°: 10 constitutes an example of embodiment of the synthetic promoter containing the seven elements listed above.

The synthetic promoter described here is intended to be used in an expression vector suitable for the stable transformation of cereal, in particular rice. In order to obtain the best results in terms of expression level, it is appropriate to fuse the sequence of the synthetic promoter with that of a 5′-UTR leader region effective in increasing the expression of recombinant proteins in plants.

The artificial DNA described here can be operatively connected to a 5′-UTR leader region effective in increasing the expression of recombinant proteins in plants and to a sequence that encodes the mature form of a heterologous protein in plants.

In particular, an appropriate expression vector comprises, in the 5′→3′ direction:
i) the endosperm-specific promoter of artificial origin, object of the present invention, upstream, that is, in position 5′, of a nucleotide sequence of natural or artificial origin encoding the mature form of a protein;
ii) the artificial DNA of the 5′-UTR leader region effective in increasing the expression of recombinant proteins in plants, interposed between the promoter and the coding sequence;
iii) a nucleotide sequence of natural or artificial origin encoding a signal peptide for sending the recombinant protein into the lumen of the endoplasmic reticulum of the cells that constitute the endosperm tissue and for its tissue accumulation;
iv) the nucleotide sequence of natural or artificial origin encoding the mature form of the protein;
v) a 3′UTR region of natural or artificial origin.

The nucleotide sequence of element ii) can for example be constructed as described in the international application WO-A-2014/111858, hereafter indicated with the initials STE, or leader STE, or alternatively the one reported in SEQ ID N°: 11 and hereafter referred to as BRU, or leader BRU.

The nucleotide sequence of element iii) can be, for example, the PSGluB4 sequence, reported in SEQ ID N°: 12 and encoding the signal peptide used in rice to convey the glutelin B4 precursor inside the endoplasmic reticulum.

The nucleotide sequence of element iv) can be the GLA sequence encoding the mature human form of the acid alpha-galactosidase enzyme (SEQ ID N°: 13). Or it can be the heavy chain or light chain sequence of an antibody, in particular an anti-TNF alpha monoclonal antibody, for example Infliximab (SEQ ID N°: 14 and SEQ ID N°: 15).

Excellent results can be obtained using the NOSter terminator of nopaline synthase as the 3′-UTR region of element v), the sequence of which is reported in SEQ ID N°: 16.

Another aspect of the present invention is also a bacterial strain containing an expression vector in accordance with the present description.

Another further aspect of the present invention are vegetable cells, plants and seeds of transformed plants obtained by means of an expression vector in accordance with the present description and also their direct or indirect use in therapeutic treatment.

### Experimental Data

Two embodiments are described below in which the synthetic promoter described here is a component of an expression vector with which a transgenic rice production line was generated: in one case it was used in a vector for the transformation of rice that contains the gene for human acid alpha-galactosidase (GLA), in another case it was used in a vector that contains the sequences encoding the heavy and light chains of an antibody, infliximab (IFX). To demonstrate the greater efficiency of the new synthetic promoter, in both cases, in parallel with the transformation with the synthetic promoter, a transformation was performed with an identical vector except for the promoter. GluB4 was used as the control promoter, the sequence of which is reported in SEQ ID N°: 17. This promoter is credited as the most effective for expression in rice endosperm (Qu and Takaiwa 2004). Its use has been described by the Applicant in international application WO-A-2009/112508 for the production of a lysosomal enzyme, and in international application WO-A-2018/189764 for the production of an antibody.

The new synthetic promoter object of the present invention was compared with GluB4 on the basis of the expression levels of the two GLA and IFX genes in rice (Oryza sativa L.), CRW3 variety. The comparison was carried out with two expression constructs, with complete equality of other elements. The plasmid pTRS, a derivative of pCAMBIA 1301 already developed by the Applicant, was used as a vector of the constructs for the stable transformation of Oryza sativa. In particular, the following pairs of vectors were compared:
pTRS/SGlbB4-STE::GLA::NOSter;
pTRS/GluB4-STE::GLA::NOSter; and
pTRS/SGlbB4-BRU::LC-IFX::NOSter/SGlbB4-BRU::HC-IFX::NOSter;
pTRS/GluB4-BRU::LC-IFX::NOSter/GluB4-BRU::HC-IFX::NOSter;

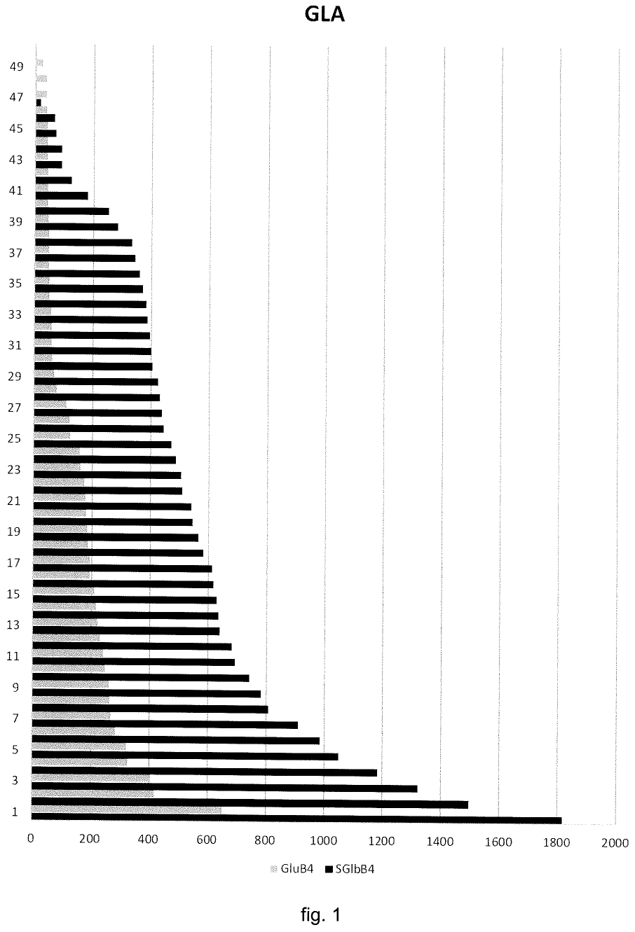

In both cases, the detection of the recombinant protein can be carried out with remarkable sensitivity and precision through an immunoassay (DAS-ELISA), as described in detail below. Each vector was inserted in Agrobacterium tumefaciensby means of electroporation for the transformation of Oryza sativa, var. CR W3 (Hiei et al. 1994). Two transgenic plant populations were obtained, each consisting of 50 individuals. The ripe seeds of each plant were collected and subjected to total protein extraction. The protein extract obtained was analyzed in DAS-ELISA to evaluate the content of acid alpha-galactosidase and infliximab. FIG. 1 shows the distribution of the data obtained for the expression of the GLA gene. The analysis of the variance allowed to establish that the differences in expression of the reporter gene found between the two rice populations considered are statistically significant, please see table 2 below.

TABLE 2
ANOVA performed on the data obtained in plants
transformed with the constructs
pTRS/SGlbB4-STE::GLA::NOSter and
pTRS/GluB4-STE::GLA::NOSter.
SUMMARY

| Groups | Count | Sum | Average | Std Deviation |
|---|---|---|---|---|
| SGlbB4 | 50 | 26088 | 522 | 382 |
| GluB4 | 50 | 7622 | 152 | 128 |

VARIANCE ANALYSIS

| Variation | | | | | Significance |
|---|---|---|---|---|---|
| origin | SQ | gdl | MQ | F | value |
| Between groups | 3409932 | 1 | 3409932 | 42 | P < 0.05 |
| Within groups | 7956241 | 98 | 81186 | | |
| Total | 11366173 | 99 | | | |

Table 2 and the graph of FIG. 1 previously mentioned show that the SGlbB4 promoter leads to expression levels certainly higher than the natural GluB4 promoter. In particular, SGlbB4 causes an increase in the expression levels of the GLA reporter gene of about 3.5 times compared to GluB4.

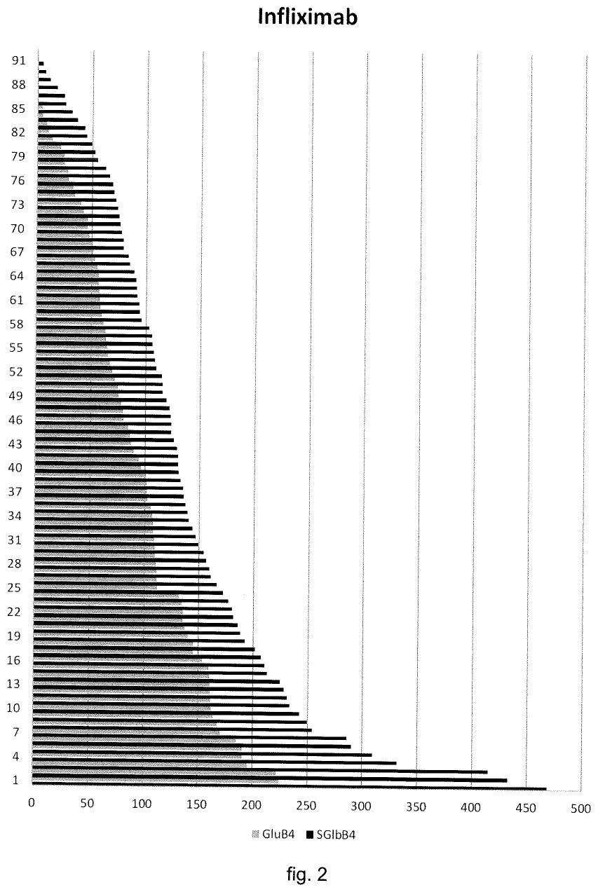

FIG. 2 shows the distribution of the data obtained for the expression of the genes for the Infliximab antibody. The variance analysis allowed to establish that the differences in the expression of the GLA reporter gene found between the two rice populations considered are statistically significant, please see table 3.

TABLE 3
ANOVA performed on the data obtained in plants
transformed with the constructs
pTRS/SGlbB4-BRU::LC-IFX::NOSter/
SGlbB4-BRU::HC-IFX::NOSter and
pTRS/GluB4-BRU::LC-IFX::NOSter/
GluB4-BRU::HC-IFX::NOSter.
SUMMARY

| Groups | Count | Sum | Average | Std Deviation |
|---|---|---|---|---|
| SGlbB4 | 92 | 12625 | 137 | 90 |
| GluB4 | 92 | 8006 | 87 | 57 |

VARIANCE ANALYSIS

| Variation | | | | | Significance |
|---|---|---|---|---|---|
| origin | SQ | gdl | MQ | F | value |
| Between groups | 115952 | 1 | 115951 | 20 | P < 0.05 |
| Within groups | 1036774 | 182 | 5697 | | |
| Total | 1152726 | 183 | | | |

Table 3 and the graph of FIG. 2 previously mentioned show that the SGlbB4 promoter leads to expression levels certainly higher than the natural GluB4 promoter. In particular, SGlbB4 causes approximately a two-fold increase in the expression levels of the antibody compared to GluB4.

### EXPERIMENTAL EXAMPLES

### Construction of the SGlbB4 Synthetic Promoter Fused to the Two STE and BRU Leaders

The GlbB4 promoter (SEQ ID N°: 8) had already been constructed previously, and was available as a pGEM-T/GlbB4 vector. The sequence of the S fragment (SEQ ID N°: 9) was artificially synthesized with an Aat II site upstream and a Sph I site downstream. The STE and BRU leader sequences (SEQ ID N°: 11) identified above were also artificially synthesized. In particular, in both cases, the synthesized segment corresponds to the sequence comprised between the Bfr I site, present in the terminal part of the rice glutelin B4 promoter (GluB4) and the Xba I site, present at 3′ of the leaders themselves.

First of all, the native leader downstream of the GluB4 promoter was replaced with the STE and BRU synthetic leaders. The starting point was the pGEM-T/GlbB4-LLTCK vector, containing the terminal part of the glutelin B4 promoter in fusion upstream with the Glb promoter and downstream with the LLTCK leader. The terminal segment of the GlbB4 promoter (from the Bfr I site) and the LLTCK leader were eliminated by means of digestion with the enzymes Bfr I and Xba I and replaced with the new synthesized sequence. In this way, the two intermediate vectors pGEM-T/GlbB4-STE and pGEM-T/GlbB4-BRU were created, subsequently verified by means of PCR analysis, enzymatic digestion and sequencing.

The S fragment (SEQ ID N°: 9) was then inserted upstream of the GlbB4 promoter. The pGEM-T/GlbB4-STE and pGEM-T/GlbB4-BRU vectors were digested with the restriction enzymes Aat II and Sph I and the S segment, extracted from pMs/S with the same enzymes, was fused by welding to obtain pGEM-T/SGlbB4-STE and pGEM-T/SGlbB4-BRU, subsequently verified by means of PCR analysis, enzymatic digestion and sequencing.

### Production of the pTRS/SGlbB4-STE::GLA::NOSter Expression Vector

The assembly of the final expression cassettes was carried out starting from the pUC18/NOSter vector. This vector was subjected to two successive sub-cloning for the insertion of the SGlbB4-STE complex and the GLA gene, respectively. The GLA gene sequence was artificially synthesized. In particular, in the first sub-cloning, pUC18/NOSter was digested with the restriction enzymes Aat II and Xba I for the welding of the SGlbB4-STE segment, extracted from the pGEM-T/SGlbB4-STE vector. In the second sub-cloning, the intermediate vector pUC18/SGlbB4-STE::NOSter was opened by means of digestion with Xba I and Sac I for the insertion of the GLA gene, extracted in turn from the pMS/GLA vector by means of the same enzymes. In this way, the pUC18 vector was obtained, containing the fully assembled expression cassettes, that is, pUC18/SGlbB4-STE::GLA::NOSter.

To produce the final vector, the SGlbB4-STE::GLA::NOSter expression cassette was extracted from pUC 18 by means of double digestion with Eco RI, and cloned into the final expression vector pCAMBIA1300/PMI in order to form: pCAMBIA1300/PMI/SGlbB4-STE::GLA::NOSter or, more briefly pTRS/SGlbB4-STE::GLA::NOSter. PRODUCTION OF THE FINAL EXPRESSION VECTOR pTRS/SGlbB4-BRU::LC-IFX::NOSter/SGlbB4-BRU::HC-IFX::NOSter

Below is an example of a procedure able to produce an expression vector that can be used in the genetic transformation of rice for the endosperm-specific expression of the Infliximab antibody in accordance with possible embodiments described here. Similar procedures can be used to obtain other antibodies or to create variants of the construct characterized by the presence of other 5′-UTR sequences and/or sequences for sending into the endoplasmic reticulum and/or terminators.

### Artificial Synthesis of the Coding Sequences

Each chain constituting the Infliximab antibody (light and heavy, abbreviated L and H, respectively) was expressed in rice through a nucleotide sequence optimized at the codon level (codon context method); both sequences encoding the light (L) and heavy (H) polypeptide chains were artificially synthesized in order to be inserted into the expression cassettes. To facilitate the plasmid cloning operations, the Xba I and Sac I restriction sites were placed at terminals 5′ and 3′. Both programmed fragments, called light chain and heavy chain respectively, once synthesized, were cloned into a specific vector.

### Production of the Molecular Expression Cassettes

The intermediate vector pUC18 (ThermoScientific) was used for the production of the expression cassettes of each Infliximab chain. More specifically, the pUC18//SGlbB4::NOS vector was used, previously produced by the Applicant, already supplied both with the SGlbB4 synthetic promoter, and also with the terminator of the nopaline synthase of Agrobacterium tumefaciens (NOS, GeneBank acc. N. AF485783). The insertion of the light chain fragment in pUC18//SGlbB4::NOS occurred by means of an oriented cloning using the Xba I and Sac I restriction sites.

The insertion of the heavy chain fragment in a second pUC18//SGlbB4::NOS was always carried out by means of oriented cloning using the Xba I and Sac I restriction sites; in this case, however, before this last step, it was necessary to modify the pUC18//SGlbB4::NOS vector. In particular, in order to promote the transfer of the expression cassette into the final vector (see next paragraph), it was necessary to replace the Eco RI restriction site, present at the 5′ terminal of the SGlbB4 promoter, with the Bgl II site. This replacement was performed by means of PCR with a specific linker design which allowed to obtain, through some intermediate steps of molecular biology and control through sequencing, the pUC18//(Bgl II) SGlbB4: : NOS vector. The latter vector was then used for the insertion of the heavy chain fragment. In this way, the two vectors carrying the expression cassettes relating to each light and heavy chain of Infliximab were constructed, namely: pUC18//SGlbB4-BRU::light chain::NOS and pUC18//(Bgl II) SGlbB4-BRU::heavy chain::NOS.

### Construction of the Final Expression Vector

For this purpose, pTRS was used, a plasmid vector developed by the Applicant, characterized by the following functional elements that are fundamental for the molecular manipulation of the expression cassettes: 1. npt II gene (present at the vector backbone level), which confers resistance to kanamycin in the selection of transformed bacterial strains; 2. PMI gene (located within the T-DNA), encoding the phospho-mannose isomerase enzyme, to be used as a positive selection agent in the early identification of transformed plants. The pTRS vector was used as an acceptor of both the expression cassettes relating to the Infliximab light and heavy chain. The cloning of the two cassettes occurred sequentially, through the Eco RI restriction sites for the light chain and the Bgl II, Mfe I pair for the heavy chain, respectively. The final expression vector was then subjected to sequence checks before being inserted by electroporation into Agrobacterium tumefaciens, strain EHA 105. The engineered Agrobacterium strain was finally used for the transformation of Oryza sativa ssp. japonica, var. CR W3.

### Genetic Transformation of Oryza sativa by means of Agrobacterium tumefaciens

The protocol of Hiei et al. (1994) was used for the genetic transformation of rice, modified by Hoge (Rice Research Group, Institute of Plant Science, Leiden University) and Guiderdoni (Biotrop program, Cirad, Montpellier, France) until the transformed calluses were obtained. For the subsequent step of selecting the transformed plant tissues, the protocol of Datta and Datta (2006) was applied, which uses as a marker system the phospho-mannose isomerase enzyme in association with increasing concentrations of mannose in the culture substrate. The main steps carried out are briefly described below.

### Preparation and Development of Embryogenic Calluses from Rice Scutellum

The transformation of rice occurred with embryogenic calluses derived from scutellum. In order to induce the proliferation of calluses from scutellum tissue, the following operating protocol was used:
the husking (removal of the glumes) of 500 rice seeds was performed;
to eliminate potential pathogens and contaminating saprophytes, disinfection of the kernels deprived of the glumes was carried out;
the first disinfection treatment provided the permanence of the seeds for 2 min in a 70% ethanol solution;
subsequently, the seeds were transferred to a 5% sodium hypochlorite solution with 2 drops of Tween-20 detergent and kept there under slow stirring for 30 min;
to eliminate all traces of sodium hypochlorite, three washes were performed with sterile H2O lasting 15 min each;
after having carried out the last washing, the seeds were dried on sterile bibulous paper;
on the surface of the callus-induction medium (CIM) substrate, dispensed in the volume of 25 mL inside Petri dishes (Ø 90 mm), 12 seeds per plate were positioned;
the plates thus obtained were incubated in the dark, at a temperature of 28° C. for 21 days; after 1 week of induction, the endosperm and the radicle were eliminated to promote the development of the callus coming from the scutellum (the scutellum is recognized by its compact mass, partly included in the yellow colored endosperm);
after 3 weeks of induction, the callus was transferred onto a renewed CIM substrate, which was followed by the fragmentation of the callous masses without the use of a scalpel, following the fraction lines naturally present on the callus;
the sub-culture continued for 10 days in order to develop the embryogenic callus and make it suitable for transformation.

### Co-culture of the Calluses with A. tumefaciens

In order to obtain sufficient quantities of A. tumefaciens for transformation, the α colony, carrying the binary expression system, was grown in liquid LB; the bacterial suspension was then distributed in Petri dishes containing LB agar-kanamycin.

After 3 days of culture at 30° C., the bacterial patina was removed and suspended in the co-cultivation medium liquid (CCML), until an O.D. 600 of about 1.0, corresponding to 3-5.109 cells/mL, was obtained.

The best calluses, that is, those with a diameter of about 2 mm, compact and whitish in color, were transferred to a Petri dish containing 35 mL of bacterial suspension and left immersed for 15 min;

We then proceeded to dry the callus using sterile bibulous paper.

A maximum number of 20 calluses was disposed per high-edge Petri dish (Sarstedt) containing the co-cultivation medium solid (CCMS).

The calluses were then incubated in the dark at a temperature of 25° C. for 3 days.

### Selection of the Calluses Transformed by Means of PMI System

After carrying out the co-culture of the embryogenic rice calluses with agrobacterium, the transformed tissues were selected, exploiting the capacity of conversion of mannose-6-phosphate into fructose-6-phosphate acquired with the insertion of the gene encoding the phospho-mannose isomerase of E. coli. The procedure used for this purpose was the following:
transfer of the calluses coming from the co-culture onto a mannose-free substrate containing 3% sucrose (SMI); incubation for 1 week in the dark at a temperature of 28° C.;
transfer of the calluses onto SMII selection substrate containing 2% sucrose and 1.5% mannose and incubation in the dark for 2 weeks at a temperature of 28° C.;
transfer of the calluses onto SMII selection substrate containing 1% sucrose and 2% mannose and incubation in the dark for 2 weeks at a temperature of 28° C.;
transfer of the calluses onto the Pre-Regeneration Medium (PRM) containing 0.5% sucrose and 2.5% mannose and incubation in the dark for 2 weeks at a temperature of 28° C.

### Regeneration of rice seedlings from transformed calluses

Non-browned calluses were transferred into high-edge Petri dishes containing the mannose-free Regeneration Medium (RM).

After 48 hours, the dishes containing the selected calluses were exposed to light.

When the seedlings were large enough to be separated from the callus (≥3 cm in height), they were transferred to culture tubes containing the rooting medium (rm).

The sub-culture inside tubes continued for about 3 weeks, always at 28° C. in the light.

At the conclusion of the regenerative process, the plants were transferred to peat and grown in greenhouses.

The composition of the various substrates used in the rice genetic transformation protocol is indicated in the two tables below.

| Composition | CIM | CCML | CCMS | PSM | SMI | SMII | PRM | RM | rm |
|---|---|---|---|---|---|---|---|---|---|
| N6 Macroelements I (mL) | 50 | | | | 50 | 50 | 50 | 50 | |
| N6 Macroelements II (mL) | 50 | | | | 50 | 50 | 50 | 50 | |
| MSFeNaEDTA (mL) | 10 | | | | 10 | 10 | 10 | 10 | |
| B5 Vitamins (mL) | 10 | | | | 10 | 10 | 10 | 10 | |
| B5 Macroelements (mL) | 1 | | | | 1 | 1 | 1 | 1 | |
| Proline (mg) | 500 | | | | 500 | 500 | 500 | 500 | |
| Glutamine (mg) | 500 | | | | 500 | 500 | 500 | 500 | |
| CEG (mg) | 300 | | | | 300 | 300 | 300 | 300 | |
| MES (mg) | 500 | | | | 500 | 500 | 500 | 500 | 500 |
| Stock RA solution (mL) | | 25 | 25 | 25 | | | | | |
| Stock RB solution (mL) | | 25 | 25 | 25 | | | | | |
| Stock RC solution (mL) | | 25 | 25 | 25 | | | | | |
| Thiamine (mg) | | 1 | 1 | 1 | | | | | 0.1 |
| 2,4-D (mg) | | 2.5 | 2.5 | 2.5 | 2.5 | 2.5 | | | |
| Glucose (mg) | | 10 | 10 | | | | | | |
| MES (mg) | | 500 | 500 | 500 | | | | | |
| Acetosyringone (mM) | | 0.1 | 0.1 | | | | | | |
| Agarose SPI (g) | | | 7 | 7 | 7 | 7 | | | |
| Phytagel (g) | | | | | | | 4.5 | 4.5 | 2.5 |
| Sucrose (g) | 30 | | | 30 | 20 | 10 | 5 | 30 | 50 |
| Mannose (g) | | | | | 15 | 20 | 25 | | |
| Cefotaxime (mg) | | | | 400 | 400 | 400 | 250 | 50 | |
| ABA (mg) | | | | | | | 5 | | |
| BAP (mg) | | | | | | | 2 | 3 | |
| NAA (mg) | | | | | | | 1 | 0.5 | |
| MS Salts (mL) | | | | | | | | | 100 |
| Glycine (mg) | | | | | | | | | 2 |
| Nicotinic acid (mg) | | | | | | | | | 0.5 |
| Pyridoxine (mg) | | | | | | | | | 0.5 |
| Inositol (mg) | | | | | | | | | 100 |
| H₂O | q.s | q.s | q.s | q.s | q.s | q.s | q.s | q.s | q.s |
| | to 1 L | to 1 L | to 1 L | to 1 L | to 1 L | to 1 L | to 1 L | to 1 L | to 1 L |

Key. CIM: Callus induction medium; CCML: co-cultivation medium liquid; CCMS: co-cultivation medium solid; PSM: pre-selection medium; SMI: selection medium I; SMII: selection medium II; PRM: pre-regeneration medium; RM: regeneration medium; rm: rooting medium.

| | Dose per L | |
|---|---|---|
| N6 macroelements II | | |
| CaCl₂2H₂O | 3.32 | g |
| N6 macroelements I | | |
| KNO₃ | 56.60 | g |
| (NH₄)SO₄ | 9.26 | g |
| KH₂PO₄ | 8.00 | g |
| MgSO₄7H₂O | 3.70 | g |
| B5 microelements | | |
| MnSO₄H₂O | 10 | g |
| KI | 0.75 | g |
| H₃BO₃ | 3 | g |
| ZnSO₄7H₂O | 2 | g |
| CuSO₄ | 0.025 | g |
| Na₂MoO₄2H₂O | 0.25 | g |
| CoCl₂6H₂O | 0.025 | g |
| B5 microelements | | |
| Inositol | 10 | g |
| Thiamine-HCL | 1 | g |
| Nicotinic acid | 0.1 | g |
| Pvridoxine-HCL | 0.1 | g |
| Stock R_{A} solution | | |
| KNO₃ | 162 | g |
| Stock R_{C} solution | | |
| CaCl₂2H₂O | 6 | g |
| H₃BO₃ | 114.4 | mg |
| Na₂MoO₄2H₂O | 5.2 | mg |
| FeSO₄7H₂O | 496 | mg |
| Na₂EDTA₂H₂O | 668 | mg |
| MS FeNaEDTA | | |
| FeSO₄7H₂O | 2.784 | g |
| Na₂EDTA₂H₂O | 3.724 | g |
| MS Salts | | |
| MnSO₄H₂O | 16.9 | mg |
| CuSO₄5H₂O | 0.025 | mg |
| ZnSO₄7H₂O | 8.63 | mg |
| CaCl₂2H₂O | 440 | mg |
| KH₂PO₄ | 170 | mg |
| KI | 0.83 | mg |
| NH₄NO₃ | 1.650 | g |
| KNO₃ | 1.9 | g |
| NaMoO₄2H₂O | 0.25 | mg |
| EDTAFeNa | 40 | mg |
| H₃BO₃ | 6.2 | mg |
| MgSO₄7H₂O | 370 | mg |
| CaCl₂6H₂O | 0.025 | mg |
| Stock R_{B} solution | | |
| MgSO₄7H₂O | 10 | g |
| (NH₄)₂SO₄ | 13.2 | g |
| NaH₂PO₄H₂O | 11 | g |
| ZnSO₄7H₂O | 88 | mg |
| MnSO₄H₂O | 80 | mg |
| CuSO₄5H₂O | 8 | mg |

### Extraction of Total Proteins from Rice Flour

The transformed rice seeds were initially husked and bleached with a Satake TO-92 bleacher (Satake Corporation, Japan). The bleached seeds were ground with a bench rotor mill (Retsch, Germany), using a 0.5 mm sieve; the resulting flour was then homogenized in extraction buffer (50 mM sodium-phosphate, 500 mM NaCl, pH 7.2), with a ratio of buffer volume (mL) to flour weight (g) equal to 5:1. After incubation at 4° C. under stirring for 1 hour, centrifugation at 3000×g was performed for 10 minutes. After the recovery of the supernatant, the residual pellet was subjected to two further extractions, with the following modifications: the ratio between extraction buffer and flour was found to be 5:1 in the second extraction and 4:1 in the third. The incubation on ice was carried out only for 10 min; after these steps, a centrifugation at 3000×g for 10 minutes always followed. The three supernatants were then combined to form a single sample used in the purification tests.

### Das-Elisa for the Detection of the Plant-Derived Infliximab Antibody

The assay was developed and applied in order to quantitatively trace the presence of the antibody in rice seeds. It was used both to validate the presence of the recombinant molecule in batches of transformed biomass, and also in the selection of primary transforms.

A 96-well, flat bottom, polystyrene plate (Costar) was subjected to coating with Goat anti-human IgG (Fc) antibody (Millipore) diluted to a concentration of 0.5 μg/mL in 2 mM sodium phosphate, 30 mM sodium chloride, pH 7.2 buffer; 100 μL of this solution was dispensed into each well. The coating process was completed in 20 min at ambient temperature, under stirring.

After removal of the coating solution, the plate was blocked for 20 min with 250 μL/well of a 1% BSA (Sigma) solution in PBS, added with 0.01% sodium azide.

After removal of the blocking solution, 50 μL of sample suitably diluted in freshly prepared PBS, 1% BSA, 0.1% Tween-20 (Sigma) were sown in each well. The samples were incubated for 20 min at 37° C. under stirring.

After removal of the samples, the wells were washed three times with freshly prepared 300 μL PBS, 0.1% Tween-20.

50 μL of Goat anti-human IgG (Fc) antibody HRP conjugate (Millipore), diluted to a concentration of 0.25 μg/mL in PBS, 1% BSA, 0.1% Tween-20, were added to each well. This was followed by an incubation of 20 min at 37° C. under stirring.

After removal of the conjugate antibody solution, the wells were washed four times with freshly prepared 300 μL PBS, 0.1% Tween-20.

100 μL TMB, liquid substrate for ELISA (ThermoFisher) were added to each well. The development occurred in about 5 min at 37° C., under stirring.

The development was stopped with 100 μL/well of 1 M hydrochloric acid. The plate was read in absorbance at a wavelength of 450 nm.

In order to construct the reference curve used in the assay, the drug Infliximab (Remicade®, Janssen Biologics B.V.) was used; the concentration range used was 2 and 40 pg/μL. The assay was therefore able to give a linear response for infliximab concentrations ranging from 0.4 to 4 ng/mL. For a good evaluation of the antibody content in the primary transforms, a 1:200 dilution of the total protein extracts obtained from the processed seed flour was used.

### Das-Elisa for the Detection of the Plant-Derived alpha-Galactosidase Enzyme

The assay was developed and applied in order to quantitatively trace the presence of the enzyme in rice seeds. It was used both to validate the presence of the recombinant molecule in batches of transformed biomass, and also in the selection of primary transforms.

A 96-well, flat bottom, polystyrene plate (Costar) was subjected to coating with a Rabbit anti-human GLA antibody (Davids biochemie) diluted to a concentration of 1 μg/mL in 2 mM sodium phosphate, 30 mM sodium chloride, pH 7.2 buffer; 100 μL of this solution was dispensed into each well. The coating process was completed in 20 min at ambient temperature, under stirring.

After removal of the coating solution, the plate was blocked for 20 min with 250 μL/well of a 1% BSA (Sigma) solution in PBS, added with 0.01% sodium azide.

After removal of the blocking solution, 50 μL of sample, suitably diluted in freshly prepared PBS, 1% BSA, 0.1% Tween-20 (Sigma), were sown in each well. The samples were incubated for 20 min at 37° C. under stirring.

After removal of the samples, the wells were washed three times with freshly prepared 300 μL PBS, 0.1% Tween-20.

50 μL of Rabbit anti-human GLA antibody HRP conjugate (Davids biochemie) diluted to a concentration of 0.25 μg/mL in PBS, 1% BSA, 0.1% Tween-20, were added to each well. This was followed by an incubation of 20 min at 37° C. under stirring.

After removal of the conjugate antibody solution, the wells were washed four times with freshly prepared 300 μL PBS, 0.1% Tween-20.

100 μL TMB, liquid substrate for ELISA (ThermoFisher) were added to each well. The development occurred in about 5 min at 37° C., under stirring.

The development was stopped with 100 μL/well of 1 M hydrochloric acid. The plate was read in absorbance at a wavelength of 450 nm.

In order to construct the reference curve used in the assay, the drug agalsidase alfa (Replagal®, Takeda) was used; the concentration range used was 12.5 and 200 ng/mL. The assay was therefore able to give a linear response for GLA concentrations ranging from 250 to 4000 ng/mL. For a good evaluation of the enzyme content in the primary transforms, a 1:20 dilution of the total protein extracts obtained from the processed seed flour was used.

### Analysis of the Primary Transforms for the Quantification of Recombinant Protein

In order to detect the presence of the alpha-galactosidase enzyme or of the Infliximab antibody in the transformed rice samples, an extraction of total proteins from 40 seeds produced by each primary transform was carried out, according to the following protocol:
grinding of each sample with a Retsch MM200 digital mill for 1 minute at a speed of 15 Hz;
recovery of the flour in an Eppendorf type test tube labelled with a specific identification code;
withdrawal of 70 mg of the flour obtained;
homogenization of the flour with 1 mL of extraction buffer in an Eppendorf type test tube labelled with a specific identification code;
transfer of the suspension into a labelled Falcon tube containing 7 mL of extraction buffer;
incubation of the tubes for 1 h on ice under stirring;
transfer of 1 mL of extract into a labelled Eppendorf type test tube and centrifugation at 16,000×g, 4° C. for 40 minutes;
recovery of the supernatant and its transfer to a new labelled Eppendorf type test tube;
storage of the remaining extract at —20° C.

This method differs from the one described above due to the presence of a single extraction intervention, but with a much lower flour weight/buffer volume ratio; by virtue of its greater simplicity, it lends itself well to the analysis of numerous samples, in particular of segregating progeny derived by self-fertilization from primary transforms. Although the procedure leads to a very diluted extract, it allows to exhaust the flour in a single step. The extract obtained is more stable and must then be diluted further in order to be analyzed with the DAS-ELISA method. This allows to evaluate the level of expression ascribable to the promoter placed upstream of the coding sequences with great accuracy.

### Purification of the Plant-Derived Infliximab Antibody

For the purification of the Infliximab antibody, a protein A based protocol was applied; all chromatographic steps were performed with AktaPurifier UPC-100 chromatograph (GE Healthcare).

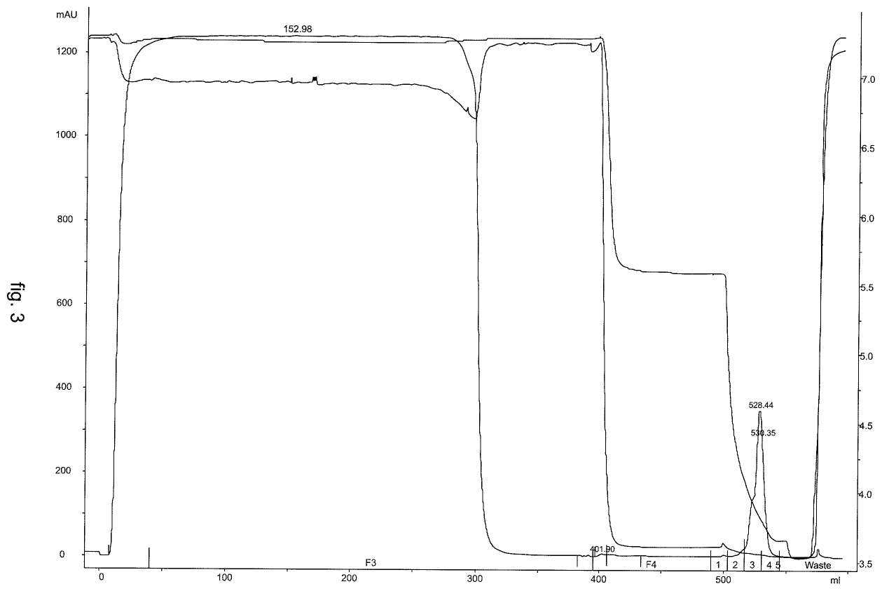

Prior to purification, the supernatant obtained from the extraction was filtered through Polycap 36 AS cartridge (Whatman), which has a porosity of 0.45 μm. The filtrate was in turn ultra-filtered with the Q-Stand tangential system (GE Healthcare), equipped with the UFP-50-C-4MA cartridge, until reaching 1/10 of the original volume. The retentate was then filtered again using a 25 mm GD/XP polyethersulfone cartridge, with a porosity of 0.45 μm (Whatman). This was followed by loading in the PRC KanCapA column (PALL); after loading, a first wash was carried out in PBS, pH 7.4, a second wash with 20 mM sodium phosphate, pH 5.5. The elution was then performed in acetate buffer (100 mM AcOH, 25 mM Arginine HCl, pH 3.5) with fraction collection of the peak (FIG. 3). The collected fractions were neutralized with 3M Tris-HCl, pH 9.1 buffer. The purified product thus obtained was used both for a biochemical characterization of the antibody molecule (study of the structure of the H and L chains), and also for an in vitro pharmacological antigen recognition test.

### Biochemical Analysis of the L and H Polypeptidic Chains of Infliximab Produced in Rice Endosperm by Means of nanoHPLC-ESI-MS/MS

A 5 μg sample of purified infliximab from rice was subjected to reduction with DTT and loaded onto 15% polyacrylamide gel (SDS-PAGE). After a short run, the gel was colored and the bands relating to the heavy and light chain were eliminated and subjected to alkylation with iodoacetamide and subsequently to proteolysis in gel. Digestion was carried out separately with trypsin, chymotrypsin, elastase, thermolysin, LysC+AspN, LysC+GluC and LysC.

Subsequently, in order to also identify the presence of the N-glycosylation site, a portion of the tryptic and chymotryptic mixture of the sample was incubated with PNGase A.

The resulting digests were analyzed by means of nanoHPLC-ESI-MS/MS with a 1100 series nanoHPLC System (Agilent Technologies, Waldbronn) and an Orbitrap XL mass spectrometer (Thermo Scientific, Bremen).

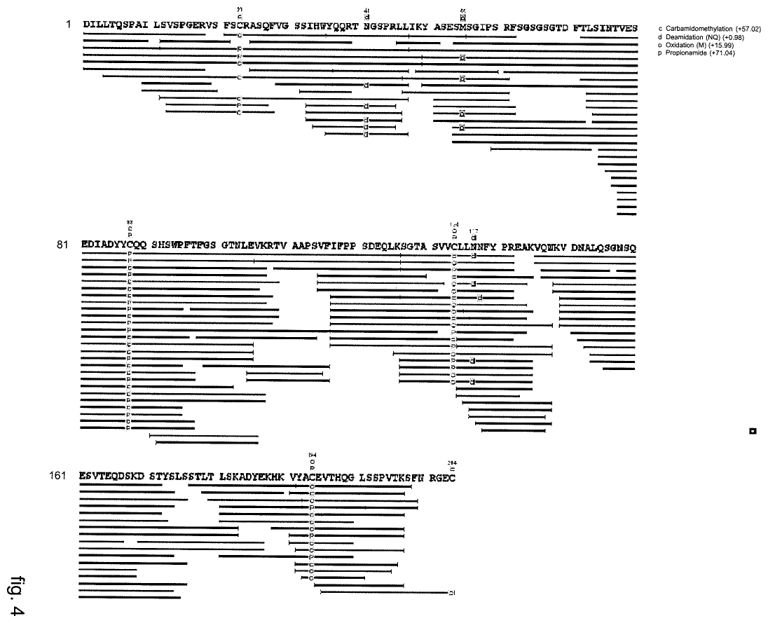

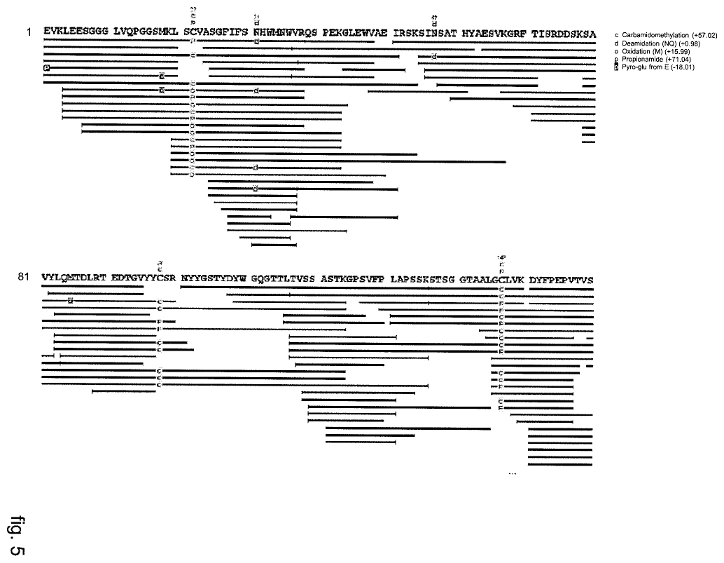

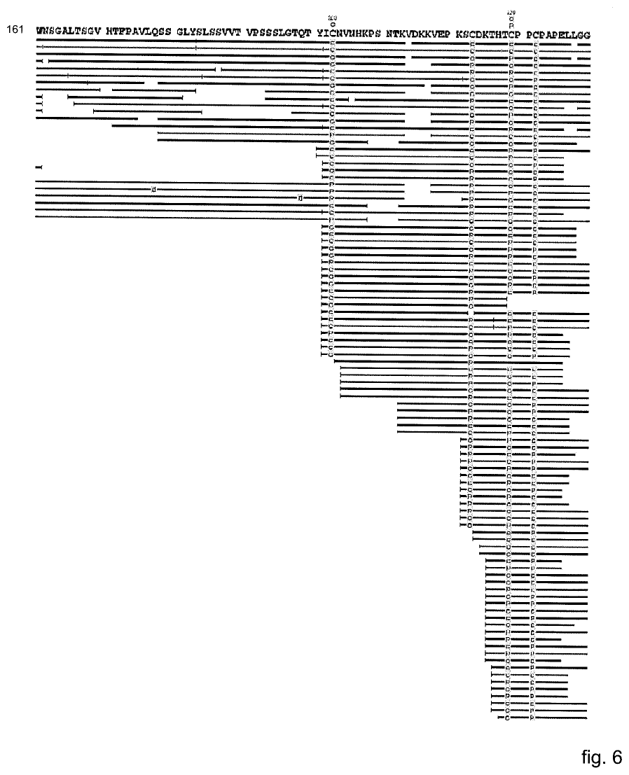

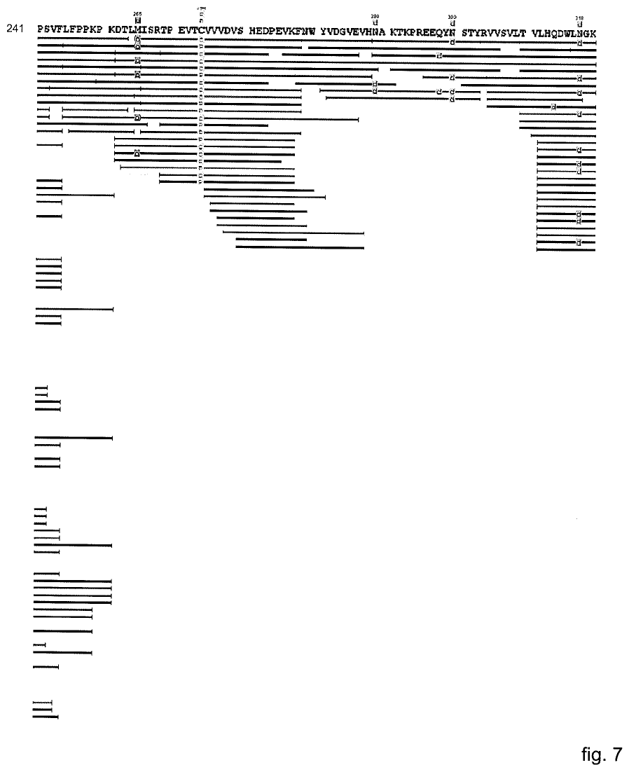

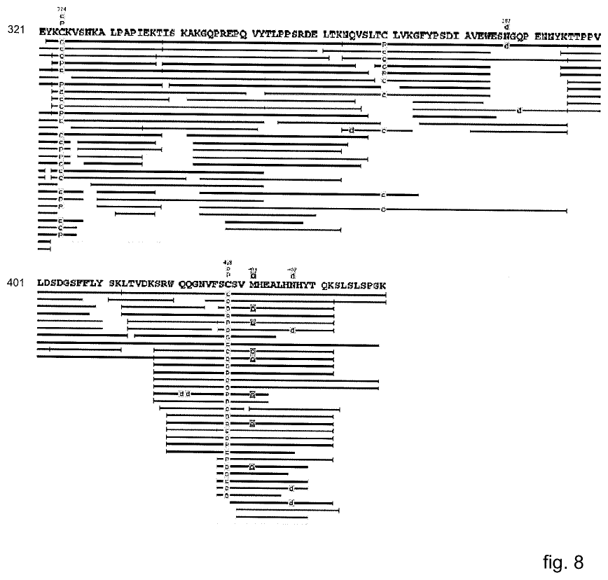

The study of the spectra obtained in the nanoHPLC-ESI-MS/MS analysis of the peptide mixtures of the two chains allowed to construct their “peptide map”. The amino acid sequence of the two chains (light and heavy) was thus confirmed, covering 100% of the sequence of both chains. For both chains, the correct removal of the signal peptide and the partial cyclization of the Gln residue in the N-terminal position were ascertained. It was also possible to identify Asn325 as an N-glycosylation site (FIGS. 4, 5, 6, 7, 8).

### Biochemical Analysis of the N-Glycosylation Profile of Infliximab Produced in Rice Endosperm by Means of nanoHPLC-ESI-MS/MS

A 5 μg sample of purified Rituximab from rice was subjected to reduction with DTT and loaded onto 15% polyacrylamide gel (SDS-PAGE). After a short run, the gel was colored and the bands relating to the heavy and light chain were eliminated and subjected to alkylation with iodoacetamide and subsequently to proteolysis in gel. Digestion was carried out with trypsin. The peptides resulting from digestion were separated and measured by means of nanoHPLC-ESI-MS/MS. To characterize the N-glycosylation profile, the spectra of the glyco-peptide variants were isolated and the integration of the area under the curve for the individual species was carried out. The processing of the spectrometric data allowed to identify 22 polysaccharide substitutions and to evaluate their relative abundance.

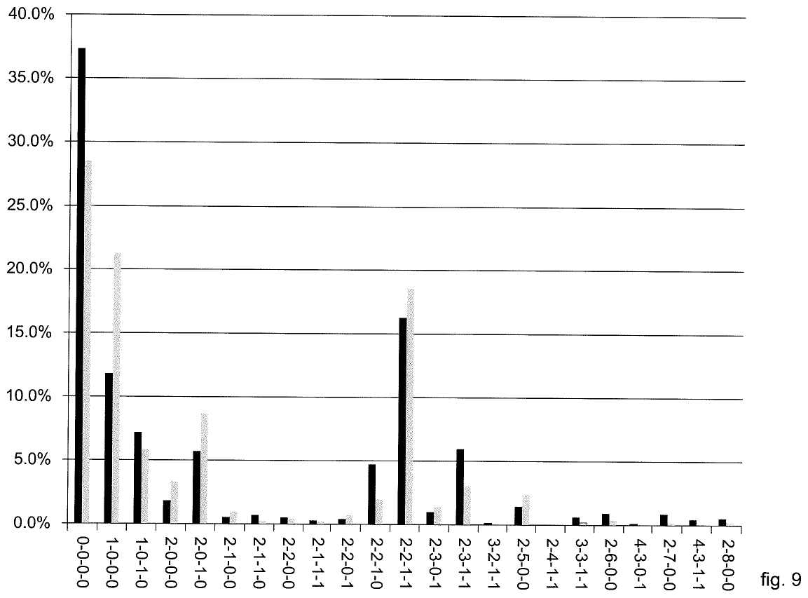

In FIG. 9, the various glycan species detected by the analysis are shown in the form of a histogram which gives an account of their relative abundance. The species are identified by four digits, for example 2-2-1-1 indicates 2 acetylglucosamines, 2 mannoses, 1 fucose and 1 xylose.

The results gathered in this experiment give a complete characterization of a rather complex glycan profile. The presence of a vegetable type glycosylation is confirmed, with short chains and the presence of characteristic monosaccharides.

In conclusion, the glycosylation profile was the one expected for proteins expressed in plants, that is, of the paucimannose type with a fucosylated core equipped, or not equipped, with a xylose residue.

It is clear that modifications and/or additions of parts may be made to the synthetic promoter for the expression of heterologous proteins in plants as described heretofore, without departing from the field and scope of the present invention, as defined by the claims.

It is also clear that, although the present invention has been described with reference to some specific examples, a person of skill in the art shall certainly be able to achieve many other equivalent forms of synthetic promoter for the expression of heterologous proteins in plants, having the characteristics as set forth in the claims and hence all coming within the field of protection defined thereby.

1. A synthetic promoter artificial DNA for use in the stable expression of Infliximab or its derivatives in rice endosperm, said artificial DNA comprising, in the 5′→3′ direction,
a plurality of DNA fragments coming from three specific natural endosperm promoters, respectively GluB4, Prol16 and Glb, operatively linked to each other by means of two synthetic linking and spacing portions interposed between said fragments, of which a first portion is an artificial sequence with a length comprised between 75 and 100 nucleotides, and a second portion is an artificial sequence with a length of at least 100 nucleotides, in particular comprised between 100 and 125 nucleotides.

2. The artificial DNA of claim 1, wherein said first linking and spacing portion is defined by SEQ ID NO: 3.

3. The artificial DNA of claim 1 , wherein said second linking and spacing portion is defined by SEQ ID NO: 5.

4. The artificial DNA of claim 1, wherein a first of said fragments, in the 5′→3′ direction, is a sequence of 82 nucleotides, present in the initial part of Glb and containing the REB binding sites for the transcription factor RISBZ2, defined by SEQ ID NO: 1.

5. The artificial DNA of claim 4, wherein a second of said fragments, operatively linked directly downstream of said first fragment, in the 5′→3′ direction, is a sequence of 208 nucleotides present in the initial part of GluB4 and defined by SEQ ID NO: 2, wherein said first linking and spacing portion is operatively linked directly downstream of said second fragment, in the 5′→3′ direction.

6. The artificial DNA of claim 1, wherein a third of said fragments, operatively linked directly downstream of said first linking portion, in the 5′→3′ direction, is a sequence coming from Prol16 and containing the two cis-regulatory motifs GCN4 and prolamin-box, wherein said second linking and spacing portion is operatively linked directly downstream of said third fragment, in the 5′→3′ direction.

7. The artificial DNA of claim 6, wherein said third fragment is a sequence of 118 nucleotides present in the intermediate part of Prol16 and defined by SEQ ID NO: 4.

8. The artificial DNA of claim 1, wherein a fourth of said fragments, operatively linked directly downstream of said second linking portion, in the 5′→3′ direction, is a sequence of 484 nucleotides that constitutes the second half of Glb and contains Glb cis-regulatory motifs, defined by SEQ ID NO: 6.

9. The artificial DNA of claim 8, wherein a fifth of said fragments, operatively linked directly downstream of said fourth fragment, in the 5′→3′ direction, is a sequence of 425 nucleotides that constitutes the final part of GluB4, including the TATA-box motif and the transcription start site, which together induce the start of mRNA synthesis on the fused DNA template downstream of the promoter, defined by SEQ ID NO: 7.

10. The artificial DNA of claim 1, defined by SEQ ID NO: 10.

11. The artificial DNA defined by SEQ ID NO: 3 or by SEQ ID NO: 5 with linking and spacing function for use in a synthetic promoter for the stable expression of Infliximab or its derivatives in rice endosperm.

12. The artificial DNA defined by SEQ ID NO: 9, with the function of enhancer for use in a synthetic promoter for the stable expression of Infliximab or its derivatives in rice endosperm.

13. An expression vector comprising the synthetic promoter artificial DNA of claim 1.

14. A method for the stable production of Infliximab or its derivatives in rice endosperm, the method comprising:
transformation of plants using the expression vector of claim 13,
industrial processing of the transformed seed, and
extraction and purification of the protein of interest.


SEQUENCE LISTING (17 sequences)

SEQ ID NO: 1 - DNA, 82 bp, Oryza sativa
  source 1..82: mol_type = genomic DNA
cgaattgccacgtaagcgctacgtcaatgctacgtcagatgaagaccgagtcaaattagccacgtaagcgccacgtcagcca

SEQ ID NO: 2 - DNA, 208 bp, Oryza sativa
  source 1..208: mol_type = genomic DNA
gtttaaacacgtgtgaccaacgtcgcggcggcagggtcaatcaagtccatggaggttatgggttccaacacagcggattggatgccgatggcacgtaactggggcgcccaatggcactcactggcctacctcaccggtcaaggtctatcctttagggtcaccaacacagatgaccaaacgctcgtcttcaccaacgtcgtgccaccag

SEQ ID NO: 3 - DNA, 89 bp, synthetic construct
  misc_feature 1..89: note = Fully synthetic portion with linking function
  source 1..89: mol_type = other DNA
gatggaagtttggccagacatttgcaagcaagctgcagttcaagtgagaggagaagcccctaggcttaagggtcacgtacccagatatc

SEQ ID NO: 4 - DNA, 89 bp, Oryza sativa; identical to SEQ ID NO: 3
  source 1..89: mol_type = genomic DNA

SEQ ID NO: 5 - DNA, 113 bp, synthetic construct
  misc_feature 1..113: note = Fully synthetic portion with linking function
  source 1..113: mol_type = other DNA
cccgggcgccgtagcggatccgctagcttaattaaggcgcgccggccggccgcgatcgccaagcatgctcccggccgccatggccgcgggattatcactagtgattgcatgcg

SEQ ID NO: 6 - DNA, 484 bp, Oryza sativa
  source 1..484: mol_type = genomic DNA
aatttgttgacaagttaagggaccttaaatgaacttattccatttcaaaatattctgtgagccatatatccgtgggcttccaatcctcctcaaattaaagggcctttttaaaatagataattgccttctttcagtcacccataaaagtacaaaactactaccaacaagcaacatgcgcagttacacacattttctgcacatttccaccacgtcacaaagagctaagagttatccctaggacaatctcattagtgtagatacatccattaatcttttatcagaggcaaacgtaaagccgctctttatgacaaaaataggtgacacaaaagtgttatctgccacatacataacttcagaaattacccaacaccaagagaaaaataaaaaaaaatctttttgcaagctccaaatcttggaaacctttttcactctttgcagcattgtactcttgctctttttccaaccgatccatgtcaccctcaag

SEQ ID NO: 7 - DNA, 425 bp, Oryza sativa
  source 1..425: mol_type = genomic DNA
cttacaagtttcttattttaaaagttccgctaacttatcaagcatagtgtgccacgcaaaactgacaacaaaccaacaaatttaaggagcgcctaacttatcatctatgacataccgcacaaaatgataacatactagagaaactttattgcacaaaaggaaatttatccataaggcaaaggaacatcttaaggctttggatatacatttaccaacaagcattgtttgtattacccctaaagcgcaagacatgtcatccatgagtcatagtgtgtatatctcaacattgcaaagctaccttttttctattatacttttcgcattataggctagatattatctatacatgtcaacaaactctatccctacgtcatatctgaagattcttttcttcactatataagttggcttccctgtcattgaac

SEQ ID NO: 8 - DNA, 610 bp, synthetic construct
  misc_feature 1..610: note = Synthetic promoter. Semi-synthetic sequence that fuses natural fragments and synthetic portions in a unique combination that has the purpose of increasing the expression level when bound to the 5 end of a specific endosperm promoter
  source 1..610: mol_type = other DNA
cgaattgccacgtaagcgctacgtcaatgctacgtcagatgaagaccgagtcaaattagccacgtaagcgccacgtcagccagtttaaacacgtgtgaccaacgtcgcggcggcagggtcaatcaagtccatggaggttatgggttccaacacagcggattggatgccgatggcacgtaactggggcgcccaatggcactcactggcctacctcaccggtcaaggtctatcctttagggtcaccaacacagatgaccaaacgctcgtcttcaccaacgtcgtgccaccaggatggaagtttggccagacatttgcaagcaagctgcagttcaagtgagaggagaagcccctaggcttaagggtcacgtacccagatatctgtctttcggaagaaccgatttactacatgactcatcaattccactttacgtatcaaaaggtttgttatgggggcaatgcttttgtgaaattaaatttttattttgcgtcacgttgtacccgggcgccgtagcggatccgctagcttaattaaggcgcgccggccggccgcgatcgccaagcatgctcccggccgccatggccgcgggattatcactagtgattgcatgcg

SEQ ID NO: 9 - DNA, 909 bp, synthetic construct
  misc_feature 1..909: note = Synthetic promoter. Semi-synthetic sequence consisting of the fusion of the proximal portions of Glb and GluB4
  source 1..909: mol_type = other DNA
aatttgttgacaagttaagggaccttaaatgaacttattccatttcaaaatattctgtgagccatatatccgtgggcttccaatcctcctcaaattaaagggcctttttaaaatagataattgccttctttcagtcacccataaaagtacaaaactactaccaacaagcaacatgcgcagttacacacattttctgcacatttccaccacgtcacaaagagctaagagttatccctaggacaatctcattagtgtagatacatccattaatcttttatcagaggcaaacgtaaagccgctctttatgacaaaaataggtgacacaaaagtgttatctgccacatacataacttcagaaattacccaacaccaagagaaaaataaaaaaaaatctttttgcaagctccaaatcttggaaacctttttcactctttgcagcattgtactcttgctctttttccaaccgatccatgtcaccctcaagcttacaagtttcttattttaaaagttccgctaacttatcaagcatagtgtgccacgcaaaactgacaacaaaccaacaaatttaaggagcgcctaacttatcatctatgacataccgcacaaaatgataacatactagagaaactttattgcacaaaaggaaatttatccataaggcaaaggaacatcttaaggctttggatatacatttaccaacaagcattgtttgtattacccctaaagcgcaagacatgtcatccatgagtcatagtgtgtatatctcaacattgcaaagctaccttttttctattatacttttcgcattataggctagatattatctatacatgtcaacaaactctatccctacgtcatatctgaagattcttttcttcactatataagttggcttccctgtcattgaac

SEQ ID NO: 10 - DNA, 1519 bp, synthetic construct
  misc_feature 1..1519: note = Synthetic super-promoter. Semi-synthetic sequence that combines the enhancer known as S and the synthetic promoter known as GlbB4
  source 1..1519: mol_type = other DNA
cgaattgccacgtaagcgctacgtcaatgctacgtcagatgaagaccgagtcaaattagccacgtaagcgccacgtcagccagtttaaacacgtgtgaccaacgtcgcggcggcagggtcaatcaagtccatggaggttatgggttccaacacagcggattggatgccgatggcacgtaactggggcgcccaatggcactcactggcctacctcaccggtcaaggtctatcctttagggtcaccaacacagatgaccaaacgctcgtcttcaccaacgtcgtgccaccaggatggaagtttggccagacatttgcaagcaagctgcagttcaagtgagaggagaagcccctaggcttaagggtcacgtacccagatatctgtctttcggaagaaccgatttactacatgactcatcaattccactttacgtatcaaaaggtttgttatgggggcaatgcttttgtgaaattaaatttttattttgcgtcacgttgtacccgggcgccgtagcggatccgctagcttaattaaggcgcgccggccggccgcgatcgccaagcatgctcccggccgccatggccgcgggattatcactagtgattgcatgcgaatttgttgacaagttaagggaccttaaatgaacttattccatttcaaaatattctgtgagccatatatccgtgggcttccaatcctcctcaaattaaagggcctttttaaaatagataattgccttctttcagtcacccataaaagtacaaaactactaccaacaagcaacatgcgcagttacacacattttctgcacatttccaccacgtcacaaagagctaagagttatccctaggacaatctcattagtgtagatacatccattaatcttttatcagaggcaaacgtaaagccgctctttatgacaaaaataggtgacacaaaagtgttatctgccacatacataacttcagaaattacccaacaccaagagaaaaataaaaaaaaatctttttgcaagctccaaatcttggaaacctttttcactctttgcagcattgtactcttgctctttttccaaccgatccatgtcaccctcaagcttacaagtttcttattttaaaagttccgctaacttatcaagcatagtgtgccacgcaaaactgacaacaaaccaacaaatttaaggagcgcctaacttatcatctatgacataccgcacaaaatgataacatactagagaaactttattgcacaaaaggaaatttatccataaggcaaaggaacatcttaaggctttggatatacatttaccaacaagcattgtttgtattacccctaaagcgcaagacatgtcatccatgagtcatagtgtgtatatctcaacattgcaaagctaccttttttctattatacttttcgcattataggctagatattatctatacatgtcaacaaactctatccctacgtcatatctgaagattcttttcttcactatataagttggcttccctgtcattgaac

SEQ ID NO: 11 - DNA, 70 bp, synthetic construct
  misc_feature 1..70: note = Artificial 5-UTR
  source 1..70: mol_type = other DNA
tcacatcaagtttccaacaacaacaacaacaacaacaacaacaacaacaacaacaaagcttctagaagcc

SEQ ID NO: 12 - DNA, 72 bp, Oryza sativa
  source 1..72: mol_type = genomic DNA
atggccaccattgcgttctcccggctgtccatctacttctgcgtgctgctgctgtgccacggctccatggcc

SEQ ID NO: 13 - DNA, 1269 bp, synthetic construct
  misc_feature 1..1269: note = Human gene rewritten for codon dialect optimization
  source 1..1269: mol_type = other DNA
atggccaccattgcgttctcccggctgtccatctacttctgcgtgctgctgctgtgccacggctccatggccctggacaatggcctggcgcggacgcccaccatgggctggctgcattgggagcggttcatgtgcaacctggactgccaggaggagccggactcctgcatctccgagaagctgttcatggagatggccgagctcatggtgtccgagggctggaaagatgccggctatgagtacctgtgcattgatgactgctggatggcgcctcagcgggactccgagggccggctgcaggccgaccctcagcggttccctcatggcatccggcagctggccaactatgtgcattccaaaggcctcaagctgggcatctatgccgatgttggcaacaagacctgcgccggcttcccgggctccttcggctactatgacattgatgcgcagaccttcgccgactggggcgttgacctgctcaagttcgatggctgctactgcgactccctggagaacctggccgatggctacaagcacatgtccctggcgctcaaccggaccggccggtccattgtgtactcctgcgagtggcctctgtacatgtggcccttccagaagcccaactacaccgagatccggcagtactgcaaccattggcggaacttcgccgacattgatgactcctggaagtccatcaagtccatcctggactggacctccttcaaccaggagcggattgttgatgttgccggcccgggcggctggaatgacccggacatgctggtgattggcaacttcggcctgtcctggaaccagcaggtgacccagatggcgctgtgggccatcatggccgcgcctctgttcatgtccaatgacctgcggcacatcagccctcaggccaaagcgctgctgcaggacaaagatgtgattgccatcaaccaggaccctctgggcaagcagggctaccagctgcggcagggcgacaacttcgaggtgtgggagcggcctctgtccggcctggcgtgggccgttgccatgatcaaccggcaggagattggcggccctcggtcctacaccattgccgttgcgtccctgggcaaaggcgttgcgtgcaacccggcgtgcttcatcacccagctgctgccggtgaagcggaagctgggcttctatgagtggacctcccggctgcggtcccacatcaatcccaccggcaccgtgctgctgcagctggagaacaccatgcagatgtccctcaaagacctgctgtag

SEQ ID NO: 14 - DNA, 1425 bp, synthetic construct
  misc_feature 1..1425: note = Chimeric human/murine gene rewritten for codon dialect optimization
  source 1..1425: mol_type = other DNA
atggccaccattgcgttctcccggctgtccatctacttctgcgtgctgctgctgtgccacggctccatggccgaggtgaagctggaggagtccggcggcggcctggtgcagccgggcggctccatgaagctgtcctgcgttgcgtccggcttcatcttctccaaccattggatgaactgggtgcggcagagcccggagaaaggcctggagtgggttgccgagatccggtccaagtccatcaactccgccacccattatgccgagtccgtgaaaggccggttcaccatctcccgggatgactccaagtccgccgtgtacctgcagatgaccgacctgcggaccgaggacaccggcgtgtactactgctcccggaactactatggctccacctatgactactggggccagggcaccaccctcaccgtgtcctccgcgtccaccaaagggccctccgtgttccctctggcgccctcctccaagtccacctccggcggcaccgccgcgctgggctgcctggtgaaagactacttcccggagccggtgaccgtgtcctggaactccggcgcgctcacctccggcgtgcacaccttcccggccgtgctgcagtcctccggcctgtactccctgtcctccgttgtgaccgtgccctcctcctccctgggcacccagacctacatctgcaatgtgaaccacaagccctccaacaccaaagttgacaagaaagttgagcccaagtcctgcgacaagacccacacctgccctccctgcccggcgccggagctgctgggcgggccctccgtgttcctgttccctcccaagcccaaagacaccctcatgatctcccggacgccggaggtgacctgcgttgttgttgatgtgtcccatgaggacccggaggtgaagttcaactggtatgttgatggcgttgaggtgcacaatgccaagaccaagcctcgggaggagcagtacaactccacctaccgggttgtgtccgtgctcaccgtgctgcaccaggactggctcaatggcaaagagtacaagtgcaaagtgtccaacaaagcgctgccggcgcccattgagaagaccatctccaaagccaaaggccagcctcgggagcctcaggtgtacaccctgcctccctcccgggatgagctgaccaagaaccaggtgtccctcacctgcctggtgaaaggcttctatccctccgacattgccgttgagtgggagtccaatggccagccggagaacaactacaagaccacgcctccggtgctggactccgatggctccttcttcctgtactccaagctcaccgttgacaagtcccggtggcagcagggcaatgtgttctcctgctccgtgatgcatgaggcgctgcacaaccattacacccagaagtccctgtccctgagcccgggcaagtga

SEQ ID NO: 15 - DNA, 717 bp, synthetic construct
  misc_feature 1..717: note = Chimeric human/murine gene rewritten for codon dialect optimization
  source 1..717: mol_type = other DNA
atggccaccattgcgttctcccggctgtccatctacttctgcgtgctgctgctgtgccacggctccatggccgacatcctgctcacccagagcccggccatcctgtccgtgagcccgggcgagcgggtgtccttctcctgccgggcgtcccagttcgttggctcctccatccattggtaccagcagcggaccaatggcagccctcggctgctcatcaagtatgcgtccgagtccatgtccggcatcccgtcccggttctccggctccggctccggcaccgacttcaccctgtccatcaacaccgttgagtccgaggacattgccgactactactgccagcagtcccattcctggcccttcaccttcggctccggcaccaacctggaggtgaagcggaccgttgccgcgccctccgtgttcatcttccctccctccgatgagcagctcaagtccggcaccgcgtccgttgtgtgcctgctcaacaacttctaccctcgggaggccaaagtgcagtggaaagttgacaatgcgctgcagtccggcaactcccaggagtccgtgaccgagcaggactccaaagactccacctactccctgtcctccaccctcacgctctccaaagccgactatgagaagcacaaagtgtatgcgtgcgaggtgacccaccagggcctgtccagcccggtgaccaagtccttcaaccgtggcgagtgctga

SEQ ID NO: 16 - DNA, 265 bp, Agrobacterium tumefaciens
  source 1..265: mol_type = genomic DNA
gaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgg

SEQ ID NO: 17 - DNA, 1430 bp, Oryza sativa
  source 1..1430: mol_type = genomic DNA
tacagggttccttgcgtgaagaagggtggcctgcggttcaccattaacggtcacgactacttccagctagtactggtgaccaacgtcgcggcggcagggtcaatcaagtccatggaggttatgggttccaacacagcggattggatgccgatggcacgtaactggggcgcccaatggcactcactggcctacctcaccggtcaaggtctatcctttagggtcaccaacacagatgaccaaacgctcgtcttcaccaacgtcgtgccaccaggatggaagtttggccagacatttgcaagcaagctgcagttcaagtgagaggagaagcctgaattgataccggagcgtttcttttgggagtaacatctctggttgcctagcaaacatatgattgtatataagtttcgttgtgcgtttattctttcggtgtgtaaaataacatacatgctttcctgatattttcttgtatatatgtacacacacacgacaaatccttccatttctattattattgaacaatttaattgcgagggcgagtacttgtctgtttaccttttttttttcagatggcattttatagtttaacctttcatggaccggcagtagttctaaccatgaatgaaaagaaatcatagtccacaccacgcagggacattgtggtcattttagacaagacgatttgattaatgtcttgtatgatatggtcgacagtgaggactaacaaacatatggcatattttattaccggcgagttaaataaatttatgtcacagtaataaactgcctaataaatgcacgccagaaaatataatgataaaaaaaagaaaagatacataagtccattgcttctacttttttaaaaattaaatccaacattttctattttttggtataaacttggaagtactagttggatatgcaaaatcatctaacctccatatatttcatcaatttgtttactttacatatgggagaggatagtatgtcaaagaaaatgacaacaagcttacaagtttcttattttaaaagttccgctaacttatcaagcatagtgtgccacgcaaaactgacaacaaaccaacaaatttaaggagcgcctaacttatcatctatgacataccgcacaaaatgataacatactagagaaactttattgcacaaaaggaaatttatccataaggcaaaggaacatcttaaggctttggatatacatttaccaacaagcattgtttgtattacccctaaagcgcaagacatgtcatccatgagtcatagtgtgtatatctcaacattgcaaagctaccttttttctattatacttttcgcattataggctagatattatctatacatgtcaacaaactctatccctacgtcatatctgaagattcttttcttcactatataagttggcttccctgtcattgaac